(19) United States
(12) Patent Application Publication
(10) Pub. No.: US 2019/0357842 A1
(43) Pub. Date: Nov. 28, 2019

(54) INFORMATION PROCESSING APPARATUS, INFORMATION PROCESSING METHOD, AND PROGRAM THEREOF

(71) Applicants: OMRON CORPORATION, Kyoto-shi (JP); OMRON HEALTHCARE CO., LTD., Muko-shi (JP)

(72) Inventors: Hiroshi USUI, Kyoto (JP); Naoki TSUCHIYA, Otsu-shi (JP)

(73) Assignees: OMRON CORPORATION, Kyoto-shi (JP); OMRON HEALTHCARE CO., LTD., Muko-shi (JP)

(21) Appl. No.: 16/536,800

(22) Filed: Aug. 9, 2019

Related U.S. Application Data
(63) Continuation of application No. PCT/JP2018/004913, filed on Feb. 13, 2018.

(30) Foreign Application Priority Data
Mar. 14, 2017 (JP) 2017-048984

(51) Int. Cl. A61B 5/00; A61B 5/021

(52) U.S. Cl. CPC A61B 5/486; A61B 5/74; A61B 5/021

(57) ABSTRACT

According to one embodiment, an apparatus includes processing circuitry coupled to a memory. The processing circuitry set a management target value related to a blood pressure feature value for each person, calculate improvement targets in lifestyle habits of the person according to the management target value, set a curved target line according to time transition data indicating a target blood-pressure lowering value at each time for predicting achievement of the management target value, detect that an actual blood-pressure lowering value obtained in time series from the person diverges from the target value, and notify the person when the divergence is detected.

### CROSS-REFERENCE TO RELATED APPLICATIONS

This application is a Continuation Application of PCT Application No. PCT/JP2018/004913, filed Feb. 13, 2018 and based upon and claiming the benefit of priority from Japanese Patent Application No. 2017-048984, filed Mar. 14, 2017, the entire contents of all of which are incorporated herein by reference.

### FIELD

The present invention relates to an information processing apparatus, an information processing method, and a program thereof, for providing support so that blood pressure falls within a proper range of a management target value based on the individual's lifestyle habits and physical condition.

### BACKGROUND

In general, high blood pressure is one of the causes of diseases such as cerebral infarction and myocardial infarction, and conversely, low blood pressure causes symptoms such as coldness and fatigue of hands and feet, which affect daily life. Therefore, it is desired to improve blood pressure to a proper level. In the case of high blood pressure, in particular, it is desired to perform blood pressure measurement every morning and evening to warn of signs of onset and to improve lifestyle habits so that the blood pressure is lowered to a proper value, and many improvement measures therefor have been proposed. For example, Patent Literature 1 (International Publication No. 2013/132696 A1) proposes a lifestyle habit improvement service provided through the Internet using a terminal device having a display screen, such as a personal computer or a smartphone. This lifestyle habit improvement service proposes a service to support improvement of lifestyle habits for lowering blood pressure to a target value by inputting a measured blood pressure and visualizing evaluation of blood pressure with use of a dedicated management application.

To date, lifestyle habit improvement services and unique applications related to blood pressure management which have been provided on the Internet perform management by setting a target based on conventional statistical data and generally-known practice. That is, differences in baseline (blood pressure status and lifestyle habits, or temperature sensitivity and salinity sensitivity, etc.) of individual blood pressure measured persons are not taken into account.

Therefore, regardless of physical and lifestyle habit differences of individual blood pressure measured persons, a conventional blood pressure management application determines a progress in blood-pressure lowering by whether it follows a uniform index or management target value based on the statistical data, without taking the differences into account. For this reason, if the blood-pressure lowering progress of the measured blood pressure deviates from the management target value, it brings about a situation where a suitable countermeasure (change of lifestyle habit improvement method) that would lead to an achievement of the management target value cannot be defined in light of the progress.

In addition, since only progress with respect to an individual management target value is notified of and the situation of others using the same blood pressure management application is not disclosed, there is no sense in sharing such among the users in a group. Thus, as can be seen in many diet treatments, it is possible that the person may feel psychologically frustrated when his/her own progress is decreased, and stop following a curriculum for blood-pressure lowering in mid-course. Furthermore, even if he/she wants to try to achieve the management target value again due to lowered progress, he/she has to perform resetting from the beginning, and thus there is a risk of giving up halfway due to the inconvenience.

Also, for a patient (blood pressure measured person) who has consulted a doctor and is having a treatment for high blood pressure, the doctor may prescribe an antihypertensive drug at the time of medical examination and instruct to measure the blood pressure at a regular time every day. However, the doctor cannot be sure that the patient will take medication and blood pressure measurements as instructed and without omission until the patient comes for the next examination.

### SUMMARY

An information processing apparatus according to the first aspect of the present invention includes: a management target value setting unit that sets a management target value related to a blood pressure feature value for each blood pressure measured person; an improvement target unit that calculates a plurality of improvement targets in lifestyle habits of the blood pressure measured person according to the management target value; an estimation unit that sets a target blood-pressure lowering line for achieving the management target value; a detection unit that detects that an actual blood-pressure lowering value obtained from the blood pressure measured person in time series diverges from the target blood-pressure lowering value by the target blood-pressure lowering line; and a communication device that notifies the blood pressure measured person when the divergence is detected.

The communication device of the information processing apparatus according to the second aspect of the present invention further transmits, to the terminal device, image information including blood pressure information including the target blood-pressure lowering value and an actual blood-pressure lowering value obtained from the blood pressure measured person in time series, and scatter range information of actually measured blood pressure values obtained from a plurality of blood pressure measured persons.

The detection unit of the information processing apparatus according to the third aspect of the present invention further compares a target blood-pressure lowering value obtained from the target blood-pressure lowering line with an actual blood-pressure lowering value obtained from the blood pressure measured person in time series, obtains a difference of a divergence between the actual blood-pressure lowering value and the target blood-pressure lowering line, and detects an occurrence of a divergence when the difference exceeds a preset threshold. The communication device further notifies the terminal device of the blood pressure measured person who corresponds to the occurrence of the divergence and the administrator terminal of the occurrence of the divergence, and suggests presence/absence of resetting of blood pressure management.

An estimation unit of an information processing apparatus according to the fourth aspect of the present invention uses the following formula as a mathematical model that predicts a degree of change in the improvement in time series:

Δy=f(Δx, t|Z_{A}, Z_{L}) (1)

where Δy: a blood pressure improvement effect, Δx: a lifestyle habit improvement amount, t: the number of elapsed days of lifestyle habit improvement (days), Z_{A}: a stratification attribute factor, Z_{L}: a stratification lifestyle habit, wherein Δy includes at least one of an average blood pressure (mmHg), variations in blood pressures measured at close times (mmHg), a difference between morning and evening blood pressures (mmHg), a night blood pressure (mmHg), and the number of times of surge blood pressures (times/night), Δx includes at least one of weight loss (kg), the number of walking steps (steps/day), an exercise amount (Ex/day), and sleep hours (minutes/day), Z_{A} includes at least one of gender, age, salinity sensitivity, temperature sensitivity, disease state, genetic inheritance, and constitution, and Z_{L} includes at least one of a body weight, an exercise amount, sleep hours, a meal amount, and a salt intake.

The information processing method according to the fifth aspect of the present invention comprises a management target value setting step for setting a management target value related to a blood pressure feature value for each blood pressure measured person, an improvement target step for calculating a plurality of improvement targets in lifestyle habits of the blood pressure measured person according to the management target value, an estimation step for setting a target blood-pressure lowering line for achieving the management target value, a detection step for detecting that the actual blood-pressure lowering value obtained from the blood pressure measured person in time series diverges from a value of the target blood-pressure lowering line, and a communication step for notifying the blood pressure measured person when the divergence is detected.

The information processing method according to the sixth aspect of the present invention further compares a target blood-pressure lowering value obtained from the target blood-pressure lowering line with the actual blood-pressure lowering value obtained from the blood pressure measured person in time series to obtain a difference of divergence between the actual blood-pressure lowering value and the target blood-pressure lowering line, detects an occurrence of the divergence when the difference exceeds a preset threshold, further notifies the terminal device of the blood pressure measured person corresponding to the occurrence of the divergence and the administrator terminal of the occurrence of the divergence, and suggests presence/absence of resetting of blood pressure management.

The information processing method according to the seventh aspect of the present invention, wherein when the occurrence of the divergence is detected, a degree of change in the improvement is lower than predicted, and it is determined that an improvement progress of the actual blood-pressure lowering value cannot be restored to the target blood-pressure lowering value by the target blood-pressure lowering line even if an improvement action for the improvement item is continued, the management target value is maintained with the actual blood-pressure lowering value at the time of the determination as an origin point, and resetting of blood pressure management to set a new target blood-pressure lowering line is performed.

According to the aspects of the present invention, it is possible to provide an information processing apparatus and an information processing method, for visualizing and presenting an expected value and an actual value of time transition of blood-pressure lowering by lifestyle habit improvement in consideration of a situation regarding a blood pressure measured person, and performs improvement so that the blood pressure becomes within a proper range of a management target value.

According to the first aspect, the information processing apparatus can perform blood pressure management reflecting physical and lifestyle habit differences of the individual blood pressure measured persons and taking into account differences in baseline (blood pressure status and lifestyle habits, or temperature sensitivity and salinity sensitivity, etc.) of the blood pressure measured persons. In addition, the information processing apparatus can give an alert and advice in a timely manner from an administrator when the progress of the blood pressure measured person is stagnated or the improvement action for the lifestyle habit improvement is diminished.

According to the second aspect, the blood pressure measured person can grasp the progress of others, and can feel the necessity of effort for achievement and obtain a sense of security as a sense of loneliness disappears.

According to the third aspect, the information processing apparatus can express a movement of a measurable index that changes in accordance with the time series according to the number of elapsed days, as a mathematical model using a relational expression.

According to the fourth aspect, the information processing method can perform blood pressure management reflecting physical and lifestyle habit differences of the individual blood pressure measured persons and taking into account differences in baseline (blood pressure status and lifestyle habits, or temperature sensitivity and salinity sensitivity, etc.) of the blood pressure measured persons.

According to the fifth aspect, the information processing method can give an alert and advice in a timely manner from an administrator when the progress of the blood pressure measured person is stagnated or the improvement action for the lifestyle habit improvement is diminished, and set a new target blood-pressure lowering line and target value of the lifestyle habit improvement with an easy operation, starting from a point at which the progress is stagnated or reduced, if the actual blood-pressure lowering value cannot be restored to the target blood-pressure lowering value even if the lifestyle habit improvement is continued.

### BRIEF DESCRIPTION OF THE DRAWINGS

FIG. 1 is a diagram showing a conceptual configuration of an information processing system including an information processing apparatus for supporting improvement in blood pressure so as to reach a management target value, according to an embodiment.

FIG. 2 is a diagram showing a configuration example of a blood pressure measuring apparatus used in the present embodiment.

FIG. 3 is a diagram showing functional blocks of the information processing apparatus, provided in a server.

FIG. 4 is a flowchart for conceptually explaining a procedure for achieving a management target value of blood pressure by lifestyle habit improvement using the information processing apparatus.

FIG. 5 is a diagram showing an example of a target blood-pressure lowering line for predicting achievement of a management target value of an average blood pressure among blood pressure improvement effects.

FIG. 6 is a diagram showing a target blood-pressure lowering line for predicting achievement of a management target value of an average blood pressure in a case of executing an improvement target.

FIG. 7 is a flowchart for explaining in detail a procedure for achieving a blood pressure management target value by lifestyle habit improvement using the information processing apparatus.

FIG. 8 is a diagram showing an actual blood-pressure lowering line of a successful example.

FIG. 9 is a diagram showing an example in which the actual blood-pressure lowering line does not follow a target blood-pressure lowering line.

FIG. 10 is a diagram showing a first display example in resetting of a target blood-pressure lowering line.

FIG. 11 is a diagram showing a second display example in resetting of a target blood-pressure lowering line.

### DETAILED DESCRIPTION

Hereinafter, embodiments of the present invention will be described in detail with reference to the drawings.

An object to be accomplished by the embodiments is to provide an information processing apparatus, an information processing method, and an information processing program, for providing support to improve blood pressure to be within a proper range of a management target value while also considering the lifestyle habit improvement amount of a blood pressure measured person in addition to the blood pressure measured by the blood pressure measured person.

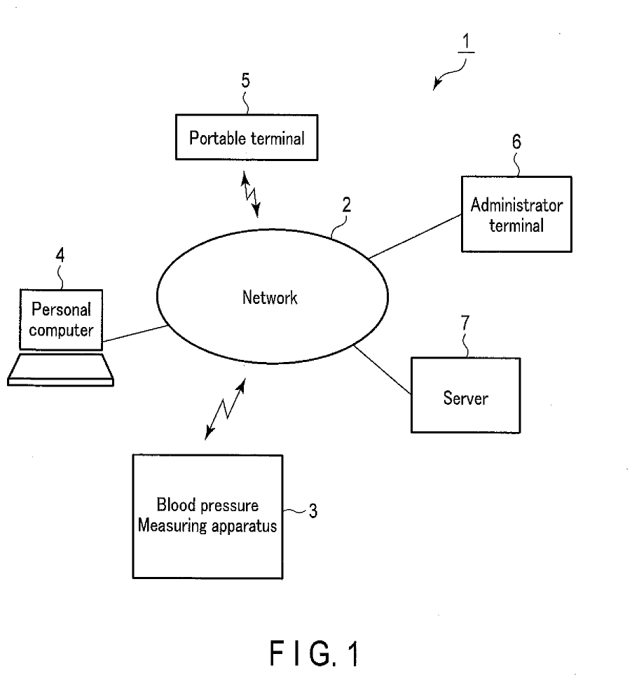

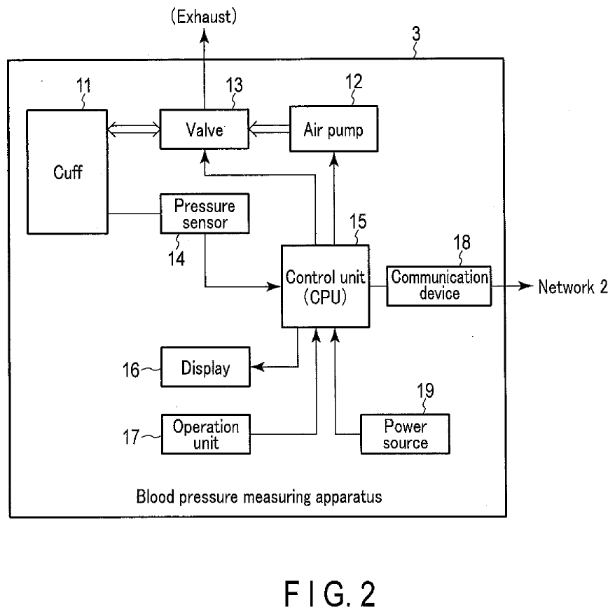

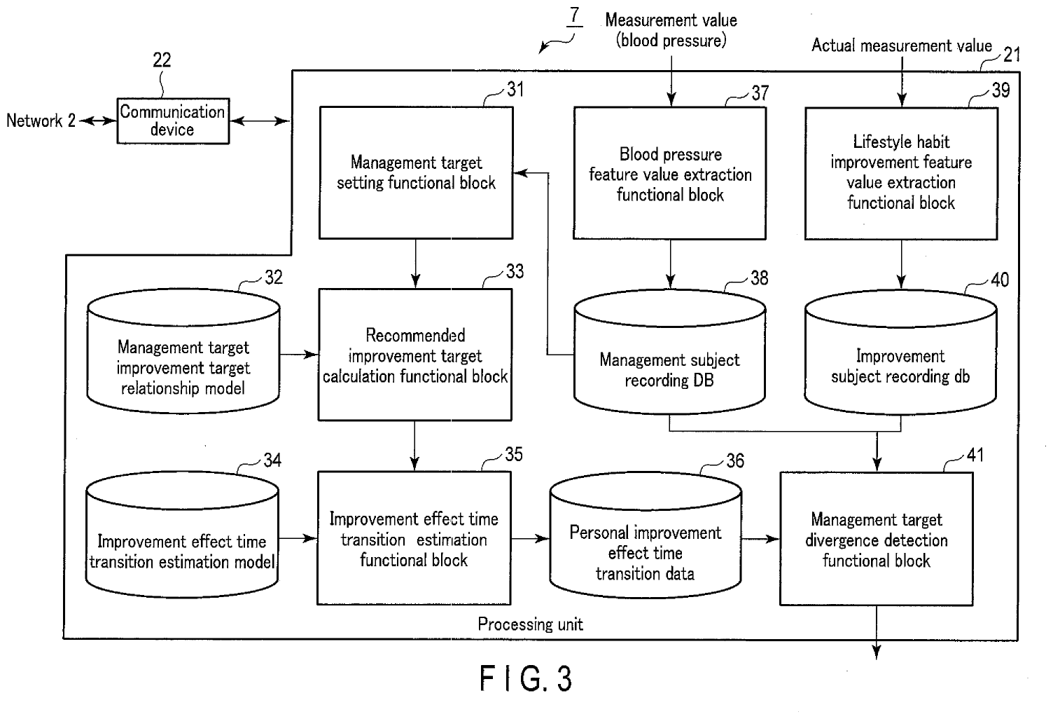

FIG. 1 is a non-limiting embodiment according to an embodiment, showing a conceptual configuration of an information processing system including an information processing apparatus for supporting to improve blood pressure so as to become a management target value. FIG. 2 is a diagram showing a configuration example of a blood pressure measuring apparatus used in the present embodiment. FIG. 3 is a diagram showing functional blocks of the information processing apparatus, provided in a server.

As shown in FIG. 1, a main configuration of an information processing system 1 of the present embodiment comprises a network 2 including the Internet, LAN, etc., a blood pressure measuring apparatus 3 to be put on a blood pressure measured person, a personal computer (hereinafter, referred to as a PC) 4 connected by wire and a portable terminal 5 such as a smartphone that can be wirelessly connected, owned by the blood pressure measured person, an administrator terminal 6 which is a terminal device disposed in a medical office or a hospital and connected wirelessly or by wire to the network 2, and a server 7 which includes an arithmetic processing unit constructing the information processing apparatus and also stores information. In the present embodiment, it is assumed that the blood pressure measured person uses the PC 4 and/or the portable terminal 5 as a terminal device, but it is not necessary to have both, and either one of them may be used. As a device necessary for network communication of a PC, such as a modem, a publicly-known device is used, and illustration and description thereof are omitted.

In FIG. 1, one of each of the portable terminal 5, the personal computer 4, and the blood pressure measuring apparatus 3 is representatively shown for ease of explanation, but in practice, it is assumed that each device is present for each of a plurality of blood pressure measured persons using this system. Herein, a person whose blood pressure is to be measured is referred to as a blood pressure measured person, but also in the case of self-measurement, the person is referred to as a blood pressure measured person. Furthermore, it is assumed that an administrator is a doctor or other medical worker who can perform a diagnosis and give advice and has a blood pressure measured person using this system under medical control. However, the administrator is not essential for operating the information processing apparatus, and the operation of the information processing apparatus is possible even in his/her absence. Herein, the blood pressure measured persons include not only a healthy person but also a person suffering from a disease and a patient being treated with or without hospitalization.

An example of the blood pressure measuring apparatus 3 will be described with reference to FIG. 2.

To the information processing system 1 of the present embodiment, for example, the blood pressure measuring apparatus 3 of either an upper arm type blood pressure measuring apparatus to be attached to an upper arm of a blood pressure measured person, or a wrist type blood pressure measuring apparatus to be worn on a wrist, can be applied. In addition, even a desk-mounted type blood pressure measuring apparatus having a cylindrical housing provided with a cuff inside can also be applied.

As shown in FIG. 2, the blood pressure measuring apparatus 3 includes a cuff 11 for applying pressure to a blood vessel of a blood pressure measured person, an air pump 12 for supplying gas (air), a valve 13 for supplying air from the air pump 12 to the cuff 11 and exhausting air from the cuff 11 to the outside, a pressure sensor 14 for measuring an internal pressure of the cuff and a blood pressure, and a control unit 15 for controlling the entire apparatus and performing blood pressure measurement. The blood pressure measuring apparatus 3 further includes a display 16 that displays detected blood pressure information, the content of an operation, etc., an operation unit 17 comprising operation buttons and a touch panel, etc. that perform measurement settings and various inputs, a communication device 18 that performs communication with other devices as shown in FIG. 1 through the network 2, and a power source 19 comprising a rechargeable battery, a primary battery, etc. Although not shown, the control unit 15 includes a memory that stores various information in a rewritable manner and a program that performs control and arithmetic processing.

In the present embodiment, an example in which the blood pressure information measured by the blood pressure measuring apparatus 3 is transmitted to the server 7 from the communication device 18 through the network 2 is indicated. However, the blood pressure measured person may key-input a blood pressure value, etc. displayed on the display 16 of the blood pressure measuring apparatus 3 from the PC 4 or the portable terminal 5, and transmit the blood pressure value, etc. to the server 7 through the network 2. Blood pressure measurement has at least two modes, a continuous measurement mode to measure during a preset period continuously, and an intermittent measurement mode (or a non-continuous measurement mode) to measure repeatedly at a set constant time period apart. Herein, also in the intermittent measurement mode, the measurement each time is to measure during a set measurement time period. Furthermore, as a communication method in the communication device 18, a general communication method, such as a Bluetooth (registered trademark) communication method, a Wi-Fi communication method, an NFC communication method, etc., can be used.

The administrator terminal 6 is formed of a PC, etc., and can count blood pressure information of a plurality of blood pressure measured persons which have been arithmetically processed by the server 7 to be described later, and can perform statistical processing. The administrator receives the blood pressure information displayed by a graph, etc. provided from the server 7, and gives an alert or advice to the blood pressure measured person who has a delay or a problem in progress. These alert and advice may be transmitted by text information through the network, or may be notified by a telephone call, an interview, or a medical examination.

Also with respect to the PC 4 or the portable terminal 5 of the blood pressure measured person, visualized statistical information, including a progress or an expected value (predicted value) for a management target value of the blood pressure measured person, and a position of the blood pressure measured person within a scatter range of other blood pressure measured persons, etc., is transmitted from the server 7.

Next, the server 7 will be described with reference to FIG. 3.

FIG. 3 shows a configuration example of functional blocks built in the server 7.

The server 7 includes a processing unit 21 having a function for performing arithmetic processing to be described later, and a communication device 22 that communicates with the blood pressure measuring apparatus 3, the portable terminal 5, and the administrator terminal 6, through the processing unit 21 and the network 2. The processing unit 21 is configured by a plurality of arithmetic circuits (or arithmetic processing units) that perform arithmetic processing in accordance with software or a program, and a plurality of memories. In the present embodiment, functions realized by software are described as functional blocks for ease of understanding. An element described as a database or a model corresponds to a storage unit. Although not shown, accompanying the server 7, the processing unit 21 includes a control circuit [control unit] that performs information processing for performing communication processing and communication with the terminal device group through the network, and controls and outputs commands to all functional blocks in an integrated manner. The information processing apparatus of the present embodiment, i.e., the processing unit 21 is a program for causing a computer to function.

The processing unit 21 includes a management target setting functional block [management target setting unit] 31, a management target/improvement target relationship model 32, a recommended improvement target calculation functional block [recommended improvement target calculation unit] 33, an improvement effect/time transition estimation model 34, an improvement effect time transition estimation functional block [improvement effect time transition estimation unit] 35, personal improvement effect time transition data 36, a blood pressure feature value extraction functional block [blood pressure feature value extraction unit] 37, management subject recording database (DB) 38, a lifestyle habit improvement feature value extraction functional block [lifestyle habit improvement feature value extraction unit] 39, improvement subject recording database (DB) 40, and a management target divergence detection functional block [management target divergence detection unit] 41.

Generally, when measuring blood-pressure lowering of high blood pressure of a blood pressure measured person, the cause of a rise in blood pressure may or may not be apparent. The causes which are apparent include, for example, in addition to stratification attribute factors (such as age, gender, salinity sensitivity and temperature sensitivity, constitution (including inheritance), etc.), a disease identified by a medical examination, a hormone secretion abnormality, and a side effect of medication, etc. The causes which are not apparent due to individual differences include, for example, a body weight/BMT, an exercise amount, sleep hours, nutrients to be ingested (e.g., diet), etc. in the stratification lifestyle habits. Improvement items of lifestyle habit improvement for high blood pressure include, but are not limited to, weight loss, increase in exercise amount, securing of sleep hours, regular time management (wake-up time and go-to-bed time, eating time, etc.), mental stability, restriction on the amount of food intake (calories), nutrient balance, etc.

Next, each of the above-described functional blocks will be described.

First, the management target setting functional block 31 is an improved target blood pressure value determined by taking into account age, gender, weight, etc. of the blood pressure measured person, in addition to an index (e.g., an average blood pressure, a night blood pressure, the number of times of surge blood pressures, a blood pressure converging time, etc.) of the blood pressure measured persons current blood pressure, and is hereinafter referred to as a management target value. Specifically, in the case of high blood pressure, this management target value is a blood pressure value that is lowered from the current blood pressure and ultimately falls within a range of a normal value for each age. In the present embodiment, although one final management target value may be used, it is also possible to set a plurality of intermediate management target values such that the blood pressure is lowered stepwise, aiming at the final management target value, so that the blood pressure measured person can obtain a sense of achievement each time.

Next, the management target/improvement target relationship model 32 will be described.

The management target/improvement target relationship model 32 is a correspondence table of a management target and an improvement target of a lifestyle habit improvement item effective for achieving the improvement target, or a mathematical model expressing their relationships. As described above, the improvement target is a target value to he achieved by improvement of a lifestyle habit so that the management target value can be achieved. The improvement target is a target value set for each improvement item in living. For example, if a means for increasing the exercise amount is a walking exercise, the improvement target is the number of walking steps per day. The target setting of the number of walking steps may be uniform, but here, as an achievable set value, the value is appropriately set by taking into consideration an exercise experience, age, or a physical condition (the presence/absence of back pain and a chronic illness related to legs and feet, etc.) in the individual blood pressure measured person.

Other improvement items for which target values can be set include matters that affect blood pressure due to daily lifestyle habits, such as a target value of weight, a target value of an exercise amount (calories consumed), sleep hours, a meal amount (nutrient balance, salinity, calories, drinking, etc.), etc.

Based on the mathematical model provided from the management target/improvement target relationship model 32, the recommended improvement target calculation functional block 33 calculates a target value of lifestyle habit improvement effective for achieving the management target value to be proposed to the blood pressure measured person.

When the management target is set by the management target setting functional block 31, the recommended improvement target calculation functional block 33 uses the management target/improvement target relationship model 32 to select an improvement item for achieving the management target, and set an improvement target corresponding to the improvement item. The blood pressure measured person may be able to modify the improvement item or the improvement target set automatically in this way.

In order to achieve the item in the lifestyle habit improvement, in the case of weight loss, for example, the blood pressure measured person may adopt a dieting method. In nutrient control, a publicly-known meal method, improvement of intake items, etc. may be adopted, in parallel with the above-described dieting. In increasing the exercise amount, there are various methods such as the above-described walking exercise, an easy stretching, and an exercise by instruction of a sports instructor, and an exercise suitable for the physical condition of the blood pressure measured person may be selected as appropriate. The settings of these target values are also possible to suit a living environment and a physical condition of the blood pressure measured person.

The improvement effect/time transition estimation model 34 is a mathematical model that predicts a degree of change in improvement in time series according to the number of elapsed days (or elapsed time) from a start in a case of achieving or maintaining the set target value of the lifestyle habit improvement. For example, it is a mathematical model that, when an average blood pressure is set as a management target value, performs the above-described lifestyle habit improvement for blood-pressure lowering, e.g., weight loss and increase in exercise amount, as improvement targets, and when the set target value (e.g., the number of steps) is maintained, e.g., predicts an effect of blood-pressure lowering of an average blood pressure along with the number of elapsed days.

Herein, an example of a mathematical model used in the management target/improvement target relationship model 32 and the improvement effect/time transition estimation model 34 will be described. In a mathematical model, the blood pressure improvement effect Δy is expressed as a function of the lifestyle habit improvement amount Δx and the number of elapsed days t of lifestyle improvement. In the blood pressure improvement effect Δy, a stratification attribute factor Z_{A} and a stratification lifestyle habit Z_{L} are also taken into consideration. In the present embodiment, this model can be expressed by the following relational expression (1).

Δy=f(Δx, t|Z_{A}, Z_{L}) (1)

where Δy: a blood pressure improvement effect, Δx: a lifestyle habit improvement amount, t: the number of elapsed days of lifestyle habit improvement (days), Z_{A}: a stratification attribute factor, and Z_{L}: a stratification lifestyle habit.

Herein, the blood pressure improvement effect Δy is, for example, at least one of average blood pressure (mmHg), variations in blood pressures measured at close times (mmHg), a difference between morning and evening blood pressures (mmHg), a night blood pressure (mmHg), and the number of times of surge blood pressures (times/night), and the lifestyle habit improvement amount Ax is at least one of weight loss (kg), the number of walking steps (steps/day), an exercise amount (Ex/day), and sleep hours (minutes/day). Also, the stratification attribute factor Z_{A} is, for example, at least one of gender, age, salinity sensitivity, and temperature sensitivity, and the stratification lifestyle habit Z_{L} is, for example, at least one of a weight, an exercise amount, and sleep hours. Furthermore, the variations in blood pressures measured at close times (mmHg) are, for example, blood pressure values obtained by performing multiple times of blood pressure measurements during a short time period in a certain hour, such as from 7 am to 8 am.

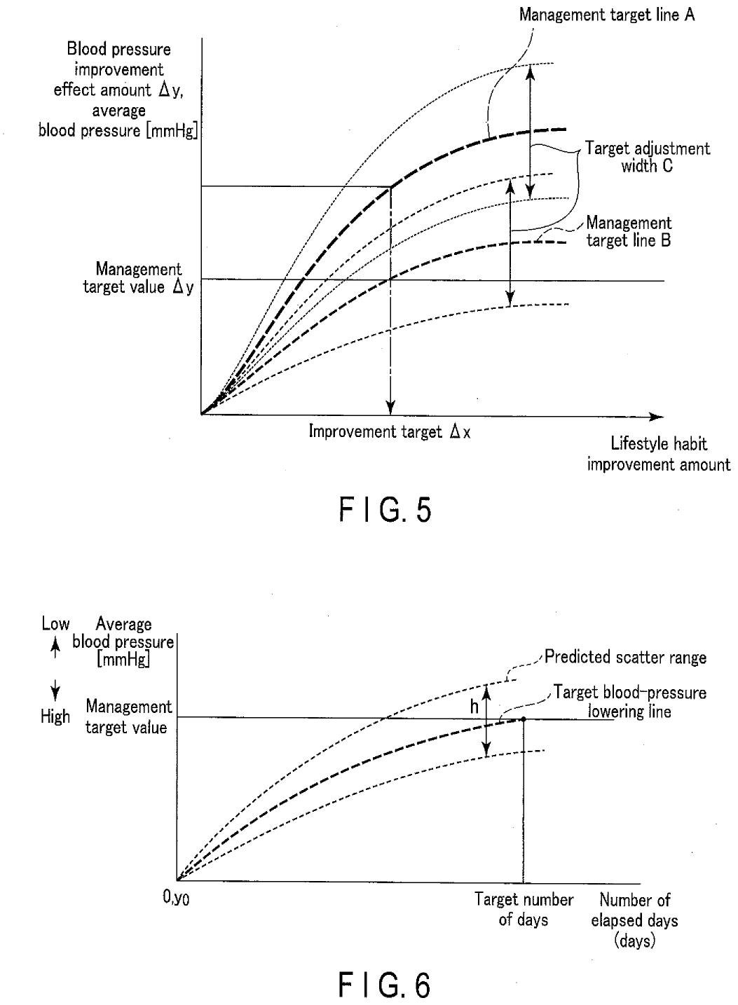

By using this relational expression (1), as shown in FIG. 5 to be described later, when each condition of Z_{A} and Z_{L} is given, by the lifestyle habit improvement amount Δx, it is possible to estimate the blood pressure improvement effect Δy of the individual blood pressure measured person. In addition, as shown in FIG. 6 to be described later, the change in state can be calculated as a change in the blood pressure improvement effect Δy according to the number of elapsed days t.

If the target value of the lifestyle habit improvement calculated by the above-described recommended improvement target calculation functional block 33 is achieved and maintained, the improvement effect time transition estimation functional block 35 estimates the number of elapsed days of the lifestyle habit improvement and the blood pressure improvement effect Δy from the relational expression (1) of the mathematical model in the improvement effect/time transition estimation model 34, and generates a target blood-pressure lowering line as shown in FIG. 6.

The personal improvement effect time transition data is a storage unit that accumulates the target blood-pressure lowering lines of the blood pressure measured person, estimated by the improvement effect time transition estimation functional block 35.

The blood pressure feature value extraction functional block 37 receives the blood pressure value measured by the blood pressure measuring apparatus 3, and performs processing of extracting, from the measured blood pressure value, and summarizing a feature value, e.g., a displacement of the blood pressure value within the measurement period, as time-series information of the blood pressure. This processing includes processing of extracting various statistics, such as an average, a variance, the maximum value, and the minimum value, in the blood pressure.

The management subject recording database (DB) 38 stores a feature value related to the blood pressure associated with the time transition output from the blood pressure feature value extraction functional block 37. This feature value is output to the management target setting functional block 31 so as to be fed back as one factor for setting a management target value.

The lifestyle habit improvement feature value extraction functional block 39 receives an actual measurement value (e.g., a measured weight) of an execution item (e.g., weight loss) of the lifestyle habit improvement performed for achieving the management target value, sets a time-series displacement of the received actual measurement value as a lifestyle habit improvement feature value, and based on the time-series displacement of the actual measurement value, performs processing of extracting and summarizing time-series information.

The improvement subject recording database (DB) 40 stores, as a database, a lifestyle habit improvement feature value associated with the time transition output from the lifestyle habit improvement feature value extraction functional block 39.

The management target divergence detection functional block 41 compares a target blood-pressure lowering value by a target blood-pressure lowering line with an actual blood-pressure lowering value obtained in time series from the blood pressure measured person, compares a difference between the actual blood-pressure lowering value and the target blood-pressure lowering value by the target blood-pressure lowering line with a preset threshold value, and if the difference exceeds the threshold value, detects that there is a divergence. The detected divergence result is transmitted from the communication device 22 to the PC 4 or the portable terminal 5 of the blood pressure measured person, and the administrator terminal 6, via the network 2.

Here, although it has been described that the processing of the processing unit 21 is performed by the server, the processing can be executed by installing a predetermined program in the PC 4 or the portable terminal 5 of the blood pressure measured person.

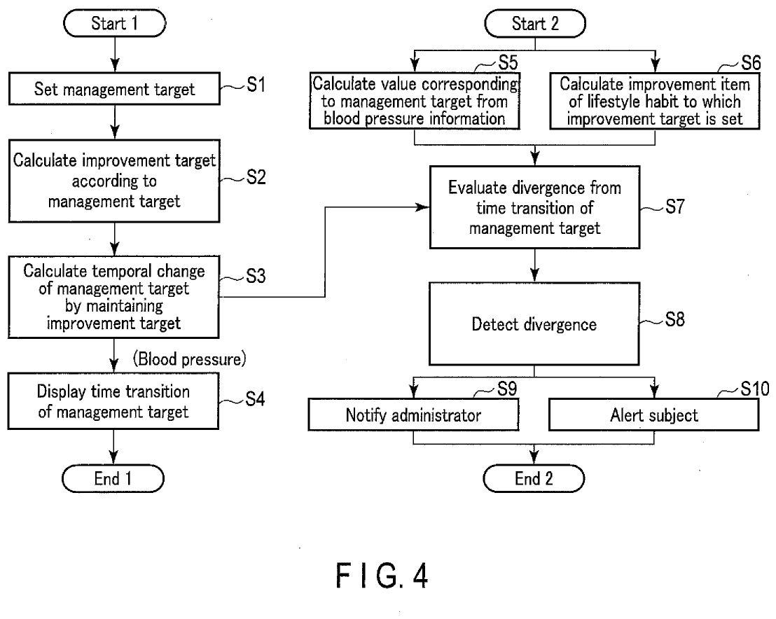

Next, a conceptual procedure for achieving a blood pressure management target value by lifestyle habit improvement using the information processing system of the present embodiment will be described with reference to a flowchart shown in FIG. 4. FIG. 5 is a diagram showing an example of an improvement target line for predicting achievement of a management target value of an average blood pressure among the blood pressure improvement effects.

First, the subject installs an application for performing processing in cooperation with the blood pressure measuring apparatus 3 and the server 7 in the portable terminal 5 (or the PC 4), and inputs personal information, current blood pressure, etc. in setting items of the application to perform initial settings. By the initial settings, information of the setting items including the above-described blood pressure information is transmitted to the server 7 via the network 2, and is input to the management target setting functional block 31. In step S1, the above-described management target value is calculated and set (management target setting functional block 31). The management target value may be set manually by a judgment of the subject or advice of the doctor.

Next, in step S2, in order to achieve the set management target value, an item (e.g., weight loss) of lifestyle habit improvement to be carried out by the blood pressure measured person and a target value (e.g., target weight) are calculated and output (recommended improvement target calculation functional block 33) using the management target and the management target/improvement target relationship model (relational expression (1)). The management target/improvement target relationship model is indicated, e.g., as shown in FIG. 5, as an example of characteristics of an improvement target and a management target line of a blood pressure improvement effect amount.

FIG. 5 is an example showing two characteristic lines of a management target line A for an obese person and a management target line B for a non-obese person, with two blood pressure measured persons having different physical conditions as an example. Thus, respectively different management target/improvement target relationship models are used to support achieving the management target value, by taking the living environment into consideration according to respective physical conditions.

For example, referring to a model in the non-obese person, as shown in FIG. 5, a target adjustment width C having a reverse-tapered expanse in the vertical direction with one management target line B at the center is shown. The target adjustment width C is an adjustment width of an expected effect according to the age, etc. in the blood pressure measured person. The blood pressure improvement effect Δy is a function of the life improvement amount Δx, and the larger the improvement target is, the larger the management target value Δy is. That is, the larger the lifestyle habit improvement amount by the blood pressure measured person is, the larger the blood pressure improvement effect is. By using this model, when a management target value Δy is set, a corresponding improvement target value Δx can be obtained.

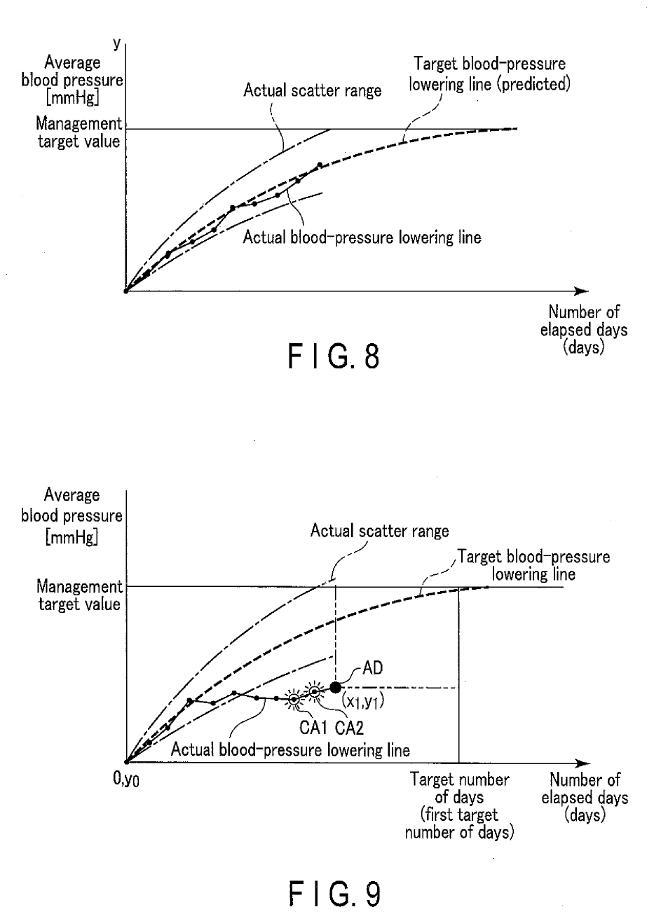

FIG. 6 is a diagram showing a target blood-pressure lowering line for predicting achievement of a management target value of an average blood pressure when an improvement target is executed. The blood pressure value becomes lower as the blood pressure value transitions up away from the origin of the vertical axis. In FIG. 6, a curved target blood-pressure lowering line passes through an intersection of the management target value and the target number of days, and a predicted scatter range h is indicated by a dotted line so as to spread on both upper and lower sides of the target blood-pressure lowering line. The predicted scatter range h indicates a prediction of a scatter range shown in FIG. 8 to be described later.

In particular, this scatter range indicates a distribution width consisting of the measured actual measurement values of all the blood pressure measured persons who use the blood pressure management application. As shown in FIG. 8, after the actual blood-pressure lowering lines are measured, the scatter range is calculated as a tabulation of the actual blood-pressure lowering lines. Within this scatter range of all the blood pressure measured persons, the position of the actual blood-pressure lowering value can be known. In addition, although not a limitation, the scatter range is used as a reference for determining the quality of the progress, based on which an alert or resetting to be described later is determined.

Next, in step 3, when the proposed improvement item and improvement target of lifestyle habits are continued, a time transition of a predicted management target (blood pressure value) approaching a management target value from as days elapse is calculated. In step S4, the time transition of the management target is displayed as a target blood-pressure lowering line of a characteristic that changes in a convex curve as shown in FIG. 6.

Next, processing of the blood pressure information measured by the blood pressure measured person and the lifestyle habit improvement amount will be described.

The blood pressure measured person executes the suggested item and the improvement target daily so as to achieve the improvement target, and performs blood pressure measurement at least once a day. The measured blood pressure information (such as a systolic blood pressure value or a diastolic blood pressure value in a predetermined period) is transmitted to the blood pressure feature value extraction functional block 37 of the server 7. At the same time, a measurement value (weight loss value, number of steps, an exercise amount, sleep hours, wake-up/going-to-bed time, ingested meal content amount, etc.) of the improvement item of the lifestyle habit improvement is transmitted to the lifestyle habit improvement feature value extraction functional block 39. It may be by key input of the portable terminal 5 or the PC 4.

In step S5, extraction or arithmetic processing is performed on the input blood pressure information to calculate values corresponding to a management target, i.e., respective actual measurement values of an average value (average blood pressure) and a variance, the maximum value (maximum blood pressure), and the minimum value (minimum blood pressure) of blood pressure in time series, and further, an actual blood-pressure lowering value is also calculated (blood pressure feature value extraction functional block 37).

In step S6, a lifestyle habit improvement amount is calculated from the input value in the improvement item for which the improvement target is set (lifestyle habit improvement feature value extraction functional block 39).

Next, in step S7, the target blood-pressure lowering line and the actual blood-pressure lowering value are compared, and a divergence is detected in step S8 (management target divergence detection functional block 41). By detecting this divergence, it is, possible to determine whether or not an improvement action of the improvement item of the lifestyle habit improvement is appropriate from FIGS. 5 and 6. For example, the larger the difference between the target blood-pressure lowering line and the actual blood-pressure lowering value is, the more the lifestyle habit improvement amount Δx is required. If the difference is equal to or greater than a predetermined value, it is determined that there is as divergence. If there is a divergence, the administrator terminal 6 is notified in step S9. At the same time, in step S10, the relevant subject is also alerted.

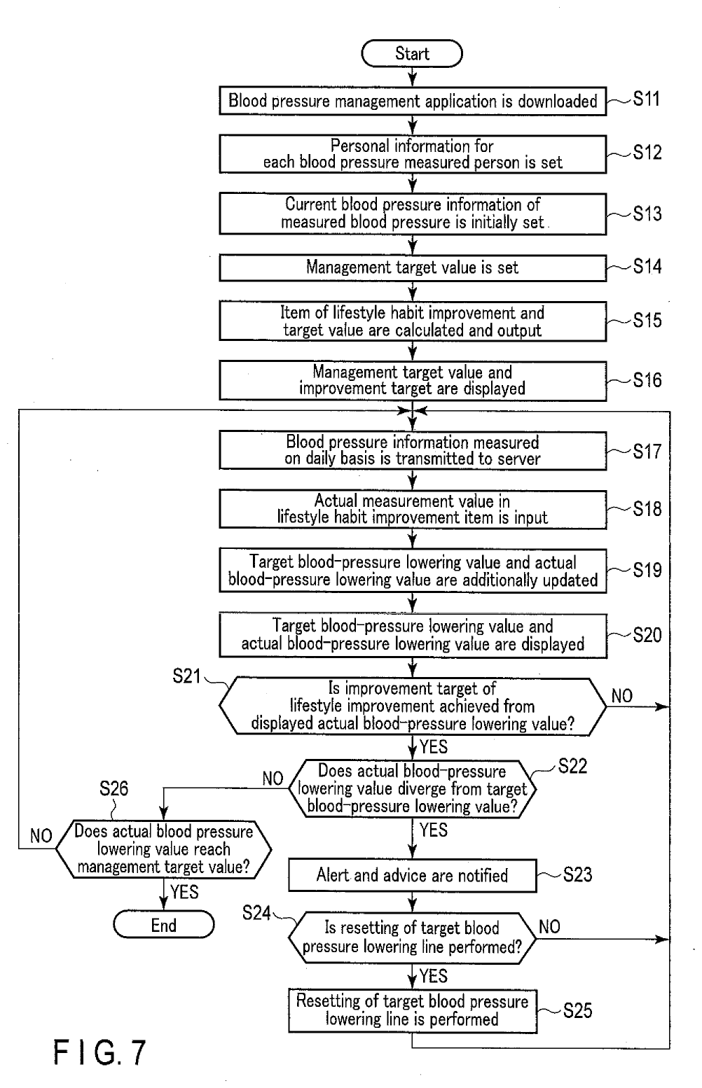

Next, with reference to the flowchart shown in FIG. 7, achieving the management target value of blood pressure by the lifestyle habit improvement using the information processing apparatus of the present embodiment will be described in detail as a series of procedures. FIGS. 5, 6, and 8 are referred to in this description.

FIG. 8 is a diagram, represented by a graph for easy visualization, showing a successful example of an actual blood-pressure lowering line that transitions in time along a predicted target blood-pressure lowering line, as a result of executing the above-described improvement target of the lifestyle habit improvement for the set management target value. Furthermore, in an upper and lower range sandwiching the actual blood-pressure lowering line, an actual scatter range indicating the distribution of all the other system users is shown, and is displayed as visualized statistical information including a position where the user is present.

First, the server 7 possessing the blood pressure management application is accessed through the network 2, the blood pressure management application is downloaded to the portable terminal 5 (or the PC 4) or read from a storage medium storing the blood pressure management application, and a transmission destination of the blood pressure measured person and information necessary for registration are input so that the blood pressure management application is set to a usable state (step S11).

Next, the blood pressure management application is activated on the portable terminal 5, and personal information for each blood pressure measured person for starting management is initialized (step S12). Specifically, in the above-described stratification attribute factors, propriety or numerical values are set to the setting items required by the blood pressure management application, such as gender and age, and further, if necessary, items such as salinity sensitivity and temperature sensitivity are also set as far as possible. Subsequently, propriety or numerical values are set to setting items, such as current weight, an exercise amount, and sleep hours, according to the lifestyle habits, as far as possible. In the blood pressure management application of the present embodiment, by inputting a large amount of personal information for the set items, an appropriate management target value can be set. Even when there is a divergence from the management target value, it is easy to find the cause, and the administrator can give appropriate advice.

Next, blood pressure measurement for initial setting is performed using the blood pressure measuring apparatus 3, and the measured current blood pressure is set as an initial value of blood pressure information in the corresponding setting item of the blood pressure management application (step S13).

Information of setting items including these blood pressure information is transmitted from the PC 4 to the server 7 via the network 2, and input to the management target setting functional block 31. In the management target setting functional block 31, the above-described management target value is calculated and set (step S14). The management target value may be set manually by a judgment of the subject or advice of the doctor. Next, in the recommended improvement target calculation functional block 33, in order to achieve the set management target value, an item (e.g., weight loss) of lifestyle habit improvement to be carried out by the blood pressure measured person and a target value (e.g., target weight) are calculated and output (step S15), and as shown in FIG. 5, a management target value of a blood pressure improvement effect Δy and an improvement target of a lifestyle habit improvement amount Δx each are visualized as a graph, etc. and displayed on the PC 4 of the blood pressure measured person. In addition to this display, the target blood-pressure lowering value shown in FIG. 6 may be displayed as another graph (step S16).

Next, the blood pressure measured person executes the improvement target daily to achieve it, and performs blood pressure measurement at least once a day. Preferably, the measurement may be performed multiple times, e.g., once at a fixed time and in the same measurement situation every morning and evening. For example, in the case of morning blood pressure measurement for the first time, it is preferable to perform the measurement after urination within 1 hour after getting up, before breakfast, and before taking medication. The second time is preferably before bedtime, after 1 hour of eating, after urination, and before bathing or after 1 hour of bathing.

In the present embodiment, the blood pressure measuring apparatus 3 has a communication function, and correctly-measured blood pressure information (the systolic blood pressure value or the diastolic blood pressure value in a predetermined period, etc.) is automatically transmitted via the network 2 to the blood pressure feature value extraction functional block 37 of the server 7 (step S17). It may be transmitted by key input of the portable terminal 5 or the PC 4. In addition, an actual measurement value (a weight loss value, the number of steps, an exercise amount, sleep hours, wake-up/going-to-bed time, ingested meal content amount, etc.) for the above-described item of lifestyle habit improvement is sent from the portable terminal 5 or the PC 4 to the lifestyle habit improvement feature value extraction functional block 39 (step S18). In this embodiment, an example is shown in which the measured blood pressure information and the measurement value of the lifestyle habit improvement item are combined and transmitted daily. However, since the lifestyle habit improvement item, such as weight, does not change daily, for example, the number of updates may be reduced by reducing the number of transmissions as compared with the blood pressure information, such as transmitting the item after an interval of three days.

Next, the blood pressure feature value extraction functional block 37 performs extraction or arithmetic processing on the input blood pressure information, and calculates respective actual measurement values of an average value (average blood pressure) and a variance, the maximum value (maximum blood pressure) and the minimum value (minimum blood pressure) of the blood pressure, in time series of the blood pressure in a fixed period in which the measurement is performed. At this time, the actual blood-pressure lowering value is calculated and output to the management subject recording database 38 so as to have the actual blood-pressure lowering value additionally updated in association with the time transition (step S19).

Next, the management target divergence detection functional block 41 reads the latest target blood-pressure lowering value and actual blood-pressure lowering value from the management subject recording database 38, and the actual blood-pressure lowering values so far are graphed and displayed as visualized statistical information on the PC 4 or the portable terminal 5 of the blood pressure measured person (step S20). At this time, the above-described actual scatter range (or an average value of a patient group having the same attribute to be described later) is also displayed.

Next, if the difference between these displayed actual blood-pressure lowering value and target blood-pressure lowering value is equal to or more than a predetermined value, the lifestyle habit improvement feature value extraction functional block 39 determines whether or not the improvement target in each item of the lifestyle habit improvement when the actual blood-pressure lowering value is calculated is achieved, through correspondence with each item of the lifestyle habit improvement input from the blood pressure measured person (step S21).

If the improvement target has not been achieved in the determination of step S21 (NO), the process returns to step S17 to continuously perform the lifestyle habit improvement and the blood pressure measurement. On the other hand, if the improvement target is achieved (YES), each shortage amount is calculated, output to the improvement subject recording database 40, and additionally updated in association with the actual blood-pressure lowering value. Thereafter, the actual blood-pressure lowering value and the target blood-pressure lowering value are compared to determine whether or not the actual blood-pressure lowering value diverges from the target blood-pressure lowering value, with respect to a predetermined determination criterion (step S22). In this determination, it is determined that there is a divergence if a difference between the actual blood-pressure lowering value and the target blood-pressure lowering value is larger than a predetermined determination criterion (difference).

In this step S22, when it is determined that the actual blood-pressure lowering value diverges from the target blood-pressure lowering value (YES), an alert that the progress is not good is notified, from the server 7 to the PC 4 or the portable terminal 5 of the blood pressure measured person, by a screen display to be described later, or the administrator gives advice for the lifestyle habit improvement to the blood pressure measured person (step S23).

Furthermore, whether or not to reset the target blood-pressure lowering line is determined based on a determination criterion of whether it is possible to reach the management target value in the target number of days predicted in advance by transitioning in the state of divergence between the actual blood-pressure lowering value and the target blood-pressure lowering value (step S24). In this determination of resetting, it is determined whether it is possible to return to the original target blood-pressure lowering value without changing the target number of days even if the progress of the current actual blood-pressure lowering value is improved. As for the target number of days, if the target number of days may be increased a little, a certain degree of allowance is used as a material for determining whether to perform resetting or not.

In the determination of step S22 described above, if the actual blood-pressure lowering value does not diverge from the target blood-pressure lowering value (NO), it is determined whether the actual blood-pressure lowering value has reached the target blood-pressure lowering value which is the same value as the management target value (step S26).

In the determination of step 826, as shown in FIG. 7, if the actual blood-pressure lowering value, i.e., the actually-measured blood pressure value, reaches the set management target value (YES), the blood pressure management application for lowering the blood pressure temporarily ends. On the other hand, if the actual blood-pressure lowering value does not reach the management target value (NO), the process returns to step S17, and lifestyle habit improvement is continued. For example, if performing blood pressure management for maintaining a blood pressure value to be measured at a management target value, it is possible to use the information processing apparatus continuously by performing resetting to be described later to reset a lifestyle habit improvement item and an improvement target value.

Next, resetting of the target blood-pressure lowering line will be described.

In the above-described flow to achieve the blood pressure management target value by the lifestyle habit improvement using the information processing apparatus, if the lifestyle habit improvement cannot be performed as planned, or if the lifestyle habit improvement is performed as instructed but the progress status is worse than predicted and the actual blood-pressure lowering line does not follow the target blood-pressure lowering line, etc., resetting of the target blood-pressure lowering line is performed. The authority to reset or not may be owned by the administrator alone, or both the administrator and the blood pressure measured person. Further, the determination of whether to reset may be either a determination by the blood pressure measured person him/herself or a determination by the administrator. The authority to determine and perform with respect to resetting is set appropriately.

In addition, a start of resetting may be configured such that a predetermined icon is displayed on a display screen of the administrator terminal 6 or a display screen of the PC 4 or the portable terminal 5 of the blood pressure measured person, and the resetting is performed by clicking the icon. When the target blood-pressure lowering line is reset, the administrator is also notified that the resetting is performed.

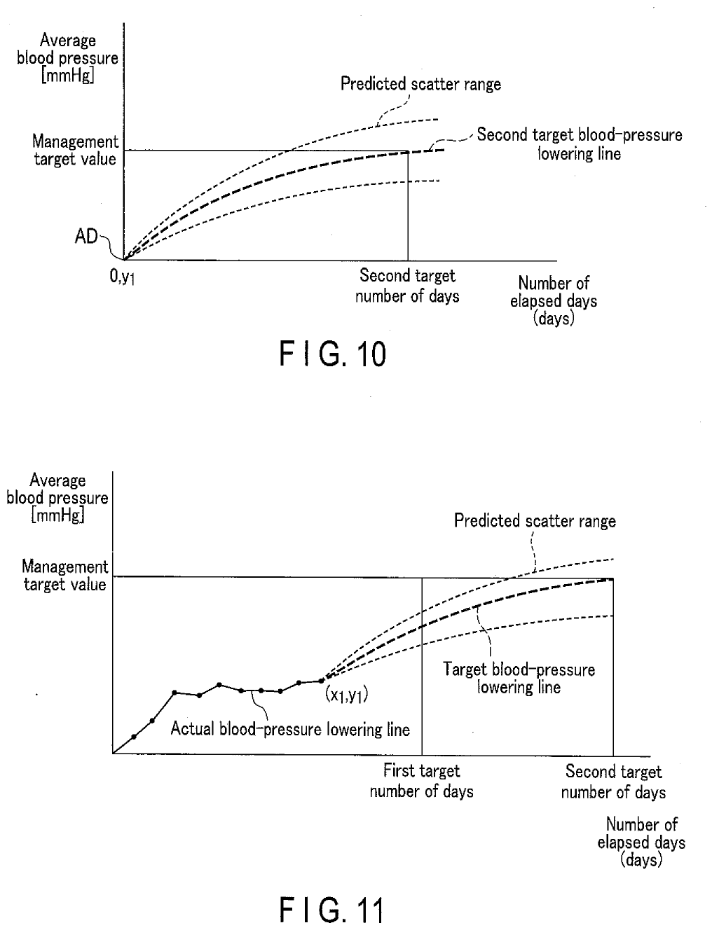

FIG. 9 is a diagram showing an example in which the actual blood-pressure lowering line does not follow the target blood-pressure lowering line. FIG. 10 is a diagram showing a first display example in resetting of a target blood-pressure lowering line, and FIG. 11 is a diagram showing a second display example in resetting of a target blood-pressure lowering line.

In FIG. 9, a lower limit line (separately, a lower limit may be set by distribution, a deviation value, etc.) in an actual scatter range by the actual blood-pressure lowering lines of all the blood pressure measured persons participating in the application of the information processing apparatus is used as a determination criterion. As an example, when a measured actual blood-pressure lowering value falls below a lower limit line in the actual scatter range, which is a lower limit determination criterion, for three consecutive days, a position CA1 of the currently measured average blood pressure value of a tip end of the actual blood-pressure lowering line on the display screen of the PC 4 or the portable terminal 5 of the corresponding blood pressure measured person changes a display color (red if a usual color is blue), and blinks.

Furthermore, if the actual blood-pressure lowering value continuously falls below the lower limit determination criterion, and in the present embodiment, the actual blood-pressure lowering value falls below the lower limit determination criterion, and the difference between the latest actual blood-pressure lowering value and a blood-pressure lowering value by the target blood-pressure lowering line exceeds a preset threshold, a position AD is changed from the blinking display to a lighting display. An alert is performed by a warning display, and blinking or lighting is raised as a display example, but a warning letter or a symbol mark may be displayed separately.

In the present embodiment, the difference between the actual blood-pressure lowering value and the blood-pressure lowering value by the target blood-pressure lowering line is used as a determination criterion, but the number of days in which the actual blood-pressure lowering value continuously falls below the lower limit determination criterion may be used as a determination criterion. This is because, if the actual blood-pressure lowering value continuously falls below the target blood-pressure lowering value, there are many cases where it may be better for the administrator to determine that there is a problem with the blood pressure measured person and deal with it as soon as possible.

When the administrator confirms this lighting display, he/she contacts and warns the corresponding blood pressure measured person. Furthermore, the administrator inquires about the current condition of the lifestyle habit improvement of the blood pressure measured person and the cause of decrease in the actual blood-pressure lowering value, and gives advice or proposes resetting of the target blood-pressure lowering line. In this explanation, the setting is that the administrator is to contact the blood pressure measured person, when the actual blood-pressure lowering value falls below the determination criterion for five consecutive days. The number of days until the administrator makes contact is set appropriately and is not limited, but if too many days pass after the actual blood-pressure lowering value starts to fall below the determination criterion, there is a concern that the blood pressure measured person may give up the lifestyle habit improvement. Therefore, it is essential to give an early alert and advice.

In resetting the target blood-pressure lowering line in this embodiment, as shown in FIG. 9, it is assumed that a management target value to be set inherits a management target value set in the previous time, and a start point at restart is a finally-measured actual blood-pressure lowering value (x1, y1). In FIG. 10, the number of elapsed days is set to an X axis, an average blood pressure is set to a Y axis, and the actual blood-pressure lowering value (x1, y1) shown in FIG. 9 is set as an origin point.

Next, a recommended improvement target is calculated from the management target value and the improvement target in the lifestyle habit improvement item, in the above-described recommended improvement target calculation functional block 33. At this time, if the cause of the actual blood-pressure lowering value falling below the determination criterion is related to the lifestyle habit improvement target, the recommended improvement target is calculated by adding a means for solving the problem.

Next, by the improvement effect time transition estimation functional block, the temporal effect on the recommended improvement target is estimated, a new target blood-pressure lowering line is predicted and reset, and a second target number of days to reach the management target value is predicted and displayed.

In the first display example shown in FIG. 10, at a graph origin point, the number of elapsed days x is set to 0, and an average blood pressure value y1, which is the actual blood-pressure lowering value obtained by lowering the blood pressure so far, is considered. For this reason, a level of reaching the management target value is low. Herein, the actual blood-pressure lowering line after resetting is displayed, and the actual blood-pressure lowering line so far is not displayed. According to this first display example, it is possible to reinvent the problems of the lifestyle habit improvement so far, and apply with new circumstances.

In the second display example of FIG. 11, a new target blood-pressure lowering line is set based on the finally-measured actual blood-pressure lowering value, but in the display, the actual blood-pressure lowering line before resetting is combined and displayed as if it were in progress. In this display, the first target number of days to be reached first is displayed, and a delay with respect to reaching the initial target is notified of. According to this second display example, by displaying the failure to achieve the target of the actual blood-pressure lowering value due to the previous lack of lifestyle habit improvement and other causes and notifying of the first failure, the negligence with respect to the lifestyle habit improvement is eliminated to help achieve a target value of each item of the lifestyle habit improvement.

As described above, according to the present embodiment, the blood pressure management reflecting physical differences and lifestyle differences of individual blood pressure measured persons and respectively taking into account differences in baseline (blood pressure status and lifestyle habits, or temperature sensitivity and salinity sensitivity, etc.) of the blood pressure measured persons, can be performed.

The information processing apparatus according to the embodiment is an application that can be easily used on a portable wireless terminal such as a smartphone, can be implemented without a fixed PC, and can be used daily even if moved to a remote place.

Since the blood pressure measuring apparatus used in the present embodiment has a communication function, the measured blood pressure can be automatically transmitted to the administrator after the measurement. When the blood pressure measured person is an elderly person, in particular, the apparatus is useful since a troublesome input operation is unnecessary, and an erroneous input by an operation of a patient can be prevented. A doctor who manages a patient with high blood pressure can also make full use of the apparatus in the diagnosis since he/she can check the progress of daily blood pressure information.

In the present embodiment, the progress with respect to the management target value for each blood pressure measured person and the actual scatter range of all of the other blood pressure measured persons are displayed together. For this reason, it is possible to confirm one's position in the whole, to have a common recognition of achieving the management target value and avoid loneliness and gain a sense of security. Conversely, it is possible to be aware of others in the group so as to raise competition and crisis awareness, and help raise the efforts for achievement, such as feeling a necessity to make more efforts.

In addition when the progress of the blood pressure measured person is stagnant or the improvement action of the lifestyle habit improvement is diminished, the administrator can give a timely alert and advice. Furthermore, starting from the point at which the progress is stagnated or lowered, new target blood-pressure lowering line and target value for lifestyle habit improvement can be set with an easy operation, so it is possible to reattempt achieving the management target value. By using these techniques, follow-up advice in order not to give up mid-way as well as perform another challenge can be realized for the blood pressure measured person.

Furthermore, if the administrator is a doctor and the blood pressure measured person is a patient having treatment for high blood pressure, during the period until the next examination, it can be confirmed daily that the patient is taking medication and measuring blood pressure without forgetting, and the status and relationship of medication and transition in blood pressure measured in the patient's home can be grasped before the examination. As a result, more effective treatment can be realized.

Furthermore, the present invention provides an information processing apparatus and an information processing method for presenting an expected value of a time transition of blood-pressure lowering and conditional probability distribution as a result of the lifestyle habit improvement, using information (age, gender, disease state, blood pressure information, life rhythm and amount, etc.) about the subject, visualizing changes in blood pressure along with the improvement of the subject, and performing improvement so that the blood pressure falls within a proper range of a management target value.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An information processing apparatus comprising processing circuitry coupled to a memory, the processing circuitry configured to:
set a management target value related to a blood pressure feature value for each blood pressure measured person;
calculate a plurality of improvement targets in lifestyle habits of the blood pressure measured person according to the management target value;
set a curved target blood-pressure lowering line according to time transition data indicating a target blood-pressure lowering value at each time for predicting achievement of the management target value corresponding to at least one of the improvement targets for each of the blood pressure measured persons;
detect that an actual blood-pressure lowering value obtained in time series from the blood pressure measured person diverges from the target blood-pressure lowering value by the target blood-pressure lowering line; and
notify the blood pressure measured person when the divergence is detected.

2. The apparatus according to claim 1, wherein the processing circuitry is further configured to notify the blood pressure measured person of image information, in which blood pressure information including the target blood-pressure lowering value and the actual blood-pressure lowering value obtained in time series from the blood pressure measured person, and scatter range information of actually measured blood pressure values obtained from the plurality of blood pressure measured persons, are included.

3. The apparatus according to claim 1, wherein the processing circuitry is further configured to:
compare the target blood-pressure lowering value obtained from the target blood-pressure lowering line with the actual blood-pressure lowering value obtained in time series from the blood pressure measured person to obtain a difference of a divergence between the actual blood-pressure lowering value and the target blood-pressure lowering line, and when the difference exceeds a preset threshold, detect an occurrence of the divergence,
notify a terminal device of a blood pressure measured person corresponding to the occurrence of the divergence and an administrator terminal of the occurrence of the divergence, and
suggest one of presence and absence of resetting of blood pressure management.

4. The apparatus according to claim 1, wherein the processing circuitry is further configured to use,
as a mathematical model that predicts a degree of change in improvement in time series,
Δy=f(Δx, t|Z_{A}, Z_{L}) (1)
where Δy: a blood pressure improvement effect, Δx: a lifestyle habit improvement amount, t: a number of elapsed days of lifestyle habit improvement (days),
ZA: a stratification attribute factor, ZL: a stratification lifestyle habit,
wherein Δy includes at least one of an average blood pressure (mmHg), a variation in blood pressures measured at close times (mmHg), a difference between morning and evening blood pressures (mmHg), a night blood pressure (mmHg), and a number of times of surge blood pressures (times/night), Δx includes at least one of weight loss (kg), a number of walking steps (steps/day), an exercise amount (Ex/day), and sleep hours (minutes/day), ZA includes at least one of gender, age, salinity sensitivity, and temperature sensitivity, and ZL includes at least one of weight, the exercise amount, and the sleep hours.

5. An information processing method comprising:
setting a management target value related to a blood pressure feature value for each blood pressure measured person,
calculating a plurality of improvement targets in lifestyle habits of the blood pressure measured person according to the management target value;
setting a curved target blood-pressure lowering line according to time transition data indicating a target blood-pressure lowering value at each time for predicting achievement of the management target value corresponding to at least one of the improvement targets for each of the blood pressure measured persons;
detecting that the actual blood-pressure lowering value obtained in time series from the blood pressure measured person diverges from a value of the target blood-pressure lowering line; and
notifying the blood pressure measured person when the divergence is detected.

6. The method according to claim 5,
wherein a target blood-pressure lowering value obtained from the target blood-pressure lowering line is compared with the actual blood-pressure lowering value obtained in time series from the blood pressure measured person to obtain a difference of a divergence between the actual blood-pressure lowering value and the target blood-pressure lowering line, and an occurrence of the divergence is detected when the difference exceeds a preset threshold and
wherein a terminal device of a blood pressure measured person corresponding to the occurrence of the divergence and an administrator terminal are notified of the occurrence of the divergence, and one of presence and absence of resetting of blood pressure management is suggested.

7. The method according to claim 5, wherein
when an occurrence of the divergence is detected, a degree of change in improvement is lower than predicted, and it is determined that an improvement progress of the actual blood-pressure lowering value cannot be restored to a target blood-pressure lowering value by the target blood-pressure lowering line even if an improvement action for an improvement item is continued, the management target value is maintained with an actual blood-pressure lowering value at a time of the determination as an origin point, and resetting of blood pressure management to set a new target blood-pressure lowering line is performed.

8. The apparatus according to claim 1, wherein the processing circuitry is further configured to, when the occurrence of the divergence is detected, a degree of change in improvement is lower than predicted, and it is determined that an improvement progress of the actual blood-pressure lowering value cannot be restored to a target blood-pressure lowering value by the target blood-pressure lowering line even if for an improvement item is continued, maintain the management target value with an actual blood-pressure lowering value of blood pressure at a time of the determination as an origin point, and perform resetting of blood pressure management to set a new target blood-pressure lowering line.

9. A non-transitory computer readable medium storing a computer program which is executed by a computer to provide the steps of:
setting a management target value related to a blood pressure feature value for each blood pressure measured person,
calculating a plurality of improvement targets in lifestyle habits of the blood pressure measured person according to the management target value;
setting a curved target blood-pressure lowering line according to time transition data indicating a target blood-pressure lowering value at each time for predicting achievement of the management target value corresponding to at least one of the improvement targets for each of the blood pressure measured persons;
detecting that the actual blood-pressure lowering value obtained in time series from the blood pressure measured person diverges from a value of the target blood-pressure lowering line; and
notifying the blood pressure measured person when the divergence is detected.

10. The computer readable medium according to claim 9, the steps further comprising:
notifying the blood pressure measured person of image information, in which blood pressure information including the target blood-pressure lowering value and the actual blood-pressure lowering value obtained in time series from the blood pressure measured person, and scatter range information of actually measured blood pressure values obtained from the plurality of blood pressure measured persons, are included.

11. The computer readable medium according to claim 9, the steps further comprising:
comparing the target blood-pressure lowering value obtained from the target blood-pressure lowering line with the actual blood-pressure lowering value obtained in time series from the blood pressure measured person to obtain a difference of a divergence between the actual blood-pressure lowering value and the target blood-pressure lowering line, and when the difference exceeds a preset threshold, detecting an occurrence of the divergence,
notifying a terminal device of a blood pressure measured person corresponding to the occurrence of the divergence and an administrator terminal of the occurrence of the divergence, and
suggesting one of presence and absence of resetting of blood pressure management.

12. The computer readable medium according to claim 9, the steps further comprising:
using as a mathematical model that predicts a degree of change in improvement in time series,
Δy=f(Δx, t|Z_{A}, Z_{L}) (1)
where Δy: a blood pressure improvement effect, ΔX: a lifestyle habit improvement amount, t: a number of elapsed days of lifestyle habit improvement (days),
ZA: a stratification attribute factor, ZL: a stratification lifestyle habit,
wherein Δy includes at least one of an average blood pressure (mmHg), a variation in blood pressures measured at close times (mmHg), a difference between morning and evening blood pressures (mmHg), a night blood pressure (mmHg), and a number of times of surge blood pressures (times/night), Δx includes at least one of weight loss (kg), a number of walking steps (steps/day), an exercise amount (Ex/day), and sleep hours (minutes/day), ZA includes at least one of gender, age, salinity sensitivity, and temperature sensitivity, and ZL includes at least one of weight, the exercise amount, and the sleep hours.

13. The computer readable medium according to claim 9, the steps further comprising:
when the occurrence of the divergence is detected, a degree of change in improvement is lower than predicted, and it is determined that an improvement progress of the actual blood-pressure lowering value cannot be restored to a target blood-pressure lowering value by the target blood-pressure lowering line even if an improvement action for an improvement item is continued, maintaining the management target value with an actual blood-pressure lowering value of blood pressure at a time of the determination as an origin point, and performing resetting of blood pressure management to set a new target blood-pressure lowering line.
